(19) United States
(12) Patent Application Publication
(10) Pub. No.: US 2023/0293072 A1
(43) Pub. Date: Sep. 21, 2023

(54) METHOD AND SYSTEM FOR DETECTING DROWSINESS OF AN INDIVIDUAL

(71) Applicants: FIPSICO, Loos (FR); CENTRE HOSPITALIER REGIONAL UNIVERSITAIRE DE LILLE, Lille Cedex (FR)

(72) Inventors: Nicolas VERA, Lambersart (FR); Stéphane RIGAUT, Marcq-en-Baroeul (FR); Régis LOGIER, Marquette lez Lille (FR); Julien DE JONCKHEERE, Lomme (FR); Mathieu JEANNE, Lille (FR); Zakaria MRANI ALAOUI, Lille (FR); Baptiste CHEVALLIER, Lille (FR)

(21) Appl. No.: 18/041,603

(22) PCT Filed: Jul. 28, 2021
(86) PCT No.: PCT/EP2021/071126

§ 371 Date: Feb. 14, 2023

(30) Foreign Application Priority Data
Aug. 17, 2020 (FR) 2008528
Aug. 17, 2020 (FR) 2008529
Aug. 17, 2020 (FR) 2008530

(51) Int. Cl. A61B 5/18; A61B 5/024; A61B 5/00

(52) U.S. Cl. CPC A61B 5/18; A61B 5/02405; A61B 5/7267

(57) ABSTRACT

A method to detect drowsiness of an individual. The method includes: (a) acquiring a cardiac signal; (b) processing the cardiac signal to detect time intervals between successive heartbeats; (c) extracting, from the time intervals, characteristic HRV variable(s) of the heart rate variability, each HRV variable being obtained from a plurality of the time intervals; (d) calculating at least one direction aggregate for the HRV variable(s), each being a variable calculated from the HRV variable in a sliding time window, and which characterizes a trend in the window, and/or calculating at least one shape aggregate for the HRV variable(s), each being calculated from values of the HRV variable in a sliding time window and quantifying the shape of a distribution of values of the HRV variable in the window; and (e) processing the direction aggregate(s) and/or the shape aggregate(s) using a detection algorithm enabling detection of drowsiness of the individual.

### TECHNICAL FIELD

The present invention relates to the detection of drowsiness from a cardiac signal of an individual. It is applicable in all fields where the detection of the drowsiness of a static individual may prove useful, and more particularly, but not exclusively, to detecting the drowsiness of a vehicle driver.

### PRIOR ART

It is estimated that between 10 and 30% of all road accidents worldwide are linked to drowsiness [NHTSA, “Drowsy Driving and Automobile Crashes”, 2017]. This estimated range is wide because the level of fatigue is not measurable after an accident, unlike for example the blood alcohol level. Thus, the investigators worked off broader criteria such as the absence of braking tracks on the ground.

Given the major problem of drowsiness at the wheel, many technical solutions have been developed to attempt to automatically detect drowsiness and alert the driver (see in particular Sahayadhas, A., Sundaraj, K., & Murugappan, M. (2012). “Detecting driver drowsiness based on sensors: a review.” Sensors, 12(12), 16937-16953.

The drowsiness of an individual can be defined in a general way as an intermediate phase of hypovigilance between an awake phase in which the individual is fully awake and vigilant, and a phase in which the individual is asleep. This intermediate phase of hypovigilance characteristic of drowsiness can itself be broken down into several successive phases of drowsiness characterized by different degrees of drowsiness.

Among known technical solutions for detecting an individual's drowsiness, a first family of detection systems based on an analysis of the behavior of the vehicle is found. In particular, a broad drowsiness detection system is one which detects crossing a white line. This system is based on the use of camera(s) to permanently analyze the characteristics of the road to determine whether the driver is not going past a white line inadvertently.

Another family of drowsiness detection systems is based on a driver behavior analysis. For this purpose, one or several cameras are used to identify the opening of the eyes [Zhang, Z.; Zhang, J. “A new real-time eye tracking based on nonlinear unscented Kalman filter for monitoring driver fatigue.” J. Contr. Theor. Appl. 2010, 8, 181-188] or the frequency of eye blinks [Bergasa, L. M.; Nuevo, J.; Sotelo, M. A.; Barea, R.; Lopez, M. E. “Real-time system formonitoring driver vigilance.” IEEE Trans. Intell. Transportation. Syst. 2006,7,63-77].

These two families have good results on paper. However, aside from the technical complexity raised by the installation of cameras on-board the vehicle, as well as the troubles due to variations in brightness, these systems are essentially reactive and can lead to drowsiness being detected too late.

It has also been sought to propose a third family of drowsiness detection systems based on measurement and analysis of the individual's physiological signals. This family is very vast and many physiological signals can be analyzed [Khushaba, R. N.; Kodagoda, S.; Lal, S.; Dissanayake, G. “Driver drowsiness classification using fuzzy wavelet-packet-based feature-extraction algorithm”. IEEE Trans. Biomed. Eng. 2011, 58, 121-131./Hu, S.; Zheng, G. “Driver drowsiness detection with eyelid related parameters by support vector machine.” Exp. Syst. Appl. 2009, 36, 7651-7658].

Among the solutions of this third family, some are based on the acquisition and analysis of an EEG (electroencephalogram) signal of the driver. Though the EEG signal may be a valuable indicator of drowsiness, it is just as problematic in several respects. From a practical point of view, the space occupied in the passenger compartment of the vehicle by such a device poses a problem. Furthermore, the data produced by the EEG signal are heavy and difficult to process in real time, and there are also problems of artifacts on the signal when the driver moves.

More recently, technical solutions for detecting drowsiness based on acquisition and analysis of a cardiac signal of the individual have been proposed, such as an ECG (electrocardiogram) signal or PPG pulse signal obtained by means of a plethysmography sensor. These solutions advantageously make it possible to implement simple, compact sensors. More particularly, sensors worn by the individual can be used, and for example sensors integrated into a bracelet or a ring. It is also possible to use sensors integrated into a garment worn by the user or sensors integrated into the driver's seat. It is also possible to use sensors integrated into the steering wheel of the vehicle.

As an individual falls asleep, a decrease in muscle tone and heart rate is observed, which represent changes in the autonomic nervous system (ANS) of the individual.

To try and detect an individual's drowsiness, it has thus been sought to use variables, commonly called HRV variables, which are characteristic of heart rate variability in the time or frequency domain and which are representative of the activity of the autonomic nervous system (ANS).

Commonly used HRV variables have, for example, been described in the publication “Heart rate variability—Standards of measurement, physiologicalinterpetation, and clinical use”, European Heart Journal, Vol. 17, March 1996, pages 354-381

The HRV variables are in a known manner calculated from a plurality of time intervals between successive heartbeats in a cardiac signal measured on an individual.

Thus, for example, in U.S. Pat. No. 9,955,925 and in U.S. patent application 2019/0008434, there are proposed systems for detecting drowsiness implementing a detection, in a cardiac signal measured on the individual, of time intervals between successive heartbeats, on an extraction of HRV variables characteristic of the variability of the heart rate, and on an analysis of these HRV variables by means of a drowsiness detection algorithm.

In the aforementioned U.S. Pat. No. 9,955,925, this detection algorithm implements an artificial neural network (ANN) trained beforehand to differentiate the individual's awake and asleep states. Detection is therefore dependent on the individual, for which the artificial neural network (ANN) has been specifically trained and is therefore not universal.

In the aforementioned U.S. patent application 2019/0008434, this detection algorithm implements a decision tree, the test variables are HRV variables in the time domain or in the frequency domain. At each node of the decision tree, at least one HRV variable is thus compared with a predefined threshold.

In both above-mentioned publications U.S. Pat. No. 9,955,925 and US 2019/0008434, the direct use of HRV variables makes these solutions less reliable, since the value of a HRV variable can be very different from one individual to the other for the same state or degree of drowsiness. Thus, as a function of the individual, the detection of drowsiness may prove to be insufficient or defective, and can in particular result in drowsiness detections coming too late to prevent the occurrence of an accident, for instance.

It has also already been proposed in the publication: Mohsen Babaeian et al: “Real time Driver Drowsiness Detection Using a Logistic-Regression-Based Machine Learning Algorithm”—IEEE Green energy and Systems Conference, Nov. 6, 2016, to calculate HRV variables called “Features”, and to use a predictive algorithm, called “Logistics regression”, to detect an individual's drowsiness.

In this publication, the predictive algorithm is applied directly to each successive instantaneous value of the HRV variable (“Feature”), and thus must necessarily be trained beforehand, in order to be adapted specifically to an individual. This is reflected in particular (cf. Table II) by a calculation, during the prior learning phase, of a specific coefficient for each individual (“Subject 1”, “Subject 2”, “Subject 3”) and for each extracted feature (left column of table II).

### Purpose of the Invention

The present invention generally aims to propose a new technical solution for detecting the drowsiness of an individual from a cardiac signal of the individual.

A more particular objective is to propose a new technical solution for detecting drowsiness that is slightly specific or slightly dependent on an individual, and which can be applied more universally to different individuals.

### SUMMARY OF THE INVENTION

The first subject of the invention is thus a method for detecting an individual's drowsiness comprising:
(a) acquiring a cardiac signal of the individual by means of at least one sensor,
(b) processing this cardiac signal allowing the detection of the time intervals (δtᵢ) between successive heartbeats,
(c) extracting, from the time intervals (δtᵢ) between successive heartbeats, one or several different characteristic HRV variables of the heart rate variability, each HRV variable being obtained from a plurality of time intervals (δtᵢ) between successive heartbeats,
(d) calculating at least one direction aggregate for one or several of the said HRV variables, each direction aggregate being a variable which is calculated from values of the HRV variable in a sliding time window (F_{Aggregate}), and which characterizes the trend (downward, upward, or constant) of the HRV variable in this sliding time window (F_{Aggregate}), and/or calculating at least one shape aggregate for one or several of the said HRV variables, each shape aggregate being a variable, which is calculated from the values of the HRV variable in a sliding time window (F_{Aggregate}) and quantifying the shape of a distribution of the values of the HRV variable in this sliding time window (F_{Aggregate}),
(e) processing the direction aggregate(s) and/or the shape aggregate(s) by a detection algorithm for detecting the individual's drowsiness.

In the context of this method of the invention, steps (c) to (e), and optionally step (b), can for example be carried out at a deferred time relative to the step (a) of acquiring the cardiac signal, by being carried out from a saved recording of this cardiac signal over a given acquisition time period. This implementation at least of steps (c) to (e) at a deferred time may prove useful for example when it is desired to detect a posteriori over a given observation period, each time period during which the individual was drowsy.

Preferably, however, steps (b) to (e) of the method of the invention are carried out during the step (a) of acquiring the cardiac signal, which allows detection of drowsiness in real time.

Optionally according to the invention, the detection method of the invention may also comprise the optional technical features below, taken in isolation or in combination:
steps (b) to (e) of the method of the invention are carried out during step (a) of acquiring the cardiac signal.
in step (c), at least two different HRV variables, preferably at least three different HRV variables, are extracted, and more preferably at least four different HRV variables.
in step (c), at least one HRV variable in the time domain and at least one HRV variable in the frequency domain are extracted.
the HRV variable(s) are chosen from the HRV variables of the following list: HRₘₑₐₙ, RMSSD, VCT, VLT, SDNN, CSI, HF, LF, HF/LF, LF/HF as defined in the description below.
in step (c), at least the variables LF and HF are extracted, and in step (d) at least one direction aggregate for each of these variables is calculated.
in step (c), at least the variables HF and HRₘₑₐₙ are extracted, and in step (d) at least one shape aggregate for each of these variables is calculated.
in step (c) several HRV variables are extracted, including at least the variable HF, and in step (d) at least one direction aggregate and at least one shape aggregate are calculated for this variable HF.
in step (c) an HR variable characteristic of the instantaneous heart rate and calculated from a single time interval (δtᵢ) between two successive heartbeats is also extracted, and in step (d) at least one direction aggregate is calculated from the values of the HR variable in a sliding time window (F_{Aggregate}), said direction aggregate characterizing the trend of the HR variable in this sliding time window (F_{Aggregate}) and/or at least one shape aggregate, from the values of this HR variable in a sliding time window (F_{Aggregate}), said shape aggregate quantifying the shape of a distribution of the values of the HR variable in this sliding time window (F_{Aggregate}).
in step (c), the HR variable and several HRV variables including at least the HF variable are extracted, and in step (d) at least one shape aggregate for each of these HR and HF variables is calculated.
a direction aggregate can more particularly be a variable whose sign defines whether the trend of the variable (HRV or HR) in said sliding time window (F_{Aggregate}) is downward or upward, preferably which is zero when the trend of the variable (HRV or HR) in said sliding time window (F_{Aggregate}) is constant, and more preferentially still whose absolute value quantifies the trend of the variable (HRV or HR) in said sliding time window (F_{Aggregate}).
at least one (“DIRECTION”) of the direction aggregates is calculated from the difference between the last and the first value of the variable in the sliding time window (F_{Aggregate}).
the sign of at least one (“DELTA”) direction aggregate is calculated from the chronological position of the maximum value of the variable in the sliding time window (F_{Aggregate}) with respect to the chronological position of said minimum value of the variable in the sliding time window (F_{Aggregate}), and preferably the absolute value of this direction aggregate (“DELTA”) is calculated from the difference between said maximum value of the variable and said minimum value of the variable.
at least one (“LINEAR REGRESSION”) of the direction aggregates is calculated from the slope of the straight line obtained by linear regression on the values of the variable in the sliding time window (F_{Aggregate}).
at least one of the shape aggregates is calculated from at least one of the following coefficients: acuity coefficient (Kurtosis) of a distribution of the variable, asymmetry coefficient (Skewness) of a distribution of the variable, standard deviation (std) of the variable.
the width of the sliding time window (F_{Aggregate}) corresponds to a time interval (T_{Aggregate}) of at least 30 seconds.
the width of the sliding time window (F_{Aggregate}) corresponds to a time interval (T_{Aggregate}) less than or equal to 10 minutes.
the width of the sliding time window (F_{Aggregate}) is adjustable.
the calculation of each direction aggregate is carried out with the same sliding time window (F_{Aggregate}).
the calculation of each shape aggregate is carried out with the same sliding time window (F_{Aggregate}).
the calculation of all the direction and/or shape aggregates is carried out with the same sliding time window (F_{Aggregate}).
the interval of the sliding time window (F_{Aggregate}) is adjustable.
step (e) comprises the use of the direction aggregate and of the shape aggregate as test variables in at least one decision tree, with an automatic classification, at the output of the decision tree, of the cardiac signal as being characteristic (S) of drowsiness of the individual or not characteristic (E) of drowsiness of the individual.
in step (c), each HRV variable is obtained in the time or frequency domain from several time intervals (δtᵢ) between successive heartbeats in a sliding time window (F_{HRV}).

Another object of the invention is a drowsiness detection system comprising a module for acquiring a cardiac signal of an individual, a module for processing the cardiac signal, adapted to perform step (b) of the aforementioned detection method, an extraction module suitable for performing step (c) of the aforementioned detection method, a calculation module suitable for performing step (d) of the aforementioned detection method, and a module for processing the direction and/or shape aggregate(s) calculated by the calculation module, which processing module is adapted to detect the drowsiness of the individual from these aggregates.

Another object of the invention is a use of the aforementioned detection system for detecting the drowsiness of an individual, and preferably the drowsiness of an individual driving a vehicle.

Another object of the invention is a computer program product comprising program code instructions and making it possible, when it is executed by one or several electronic processing units, to perform at least step (d), and preferably at least steps (d) and (e), of the aforementioned detection method.

Preferably, said computer program product makes it possible, when executed by one or several electronic processing units, to also carry out steps (a) to (c) of the aforementioned detection method.

Preferably, said computer program product makes it possible, when executed by one or several electronic processing units, to calculate, in step (c), each HRV variable in the time or frequency domain from several time intervals (δtᵢ) between successive heartbeats in a sliding time window (F_{HRV}).

### BRIEF DESCRIPTION OF THE FIGURES

The features and advantages of the invention will become more clearly apparent on reading the detailed description below of several alternative embodiments of the invention, a detailed description that is given by way of non-limiting and non-exhaustive example of the invention, and with reference to the appended drawings, in which:

FIG. 1 shows an example detection system of the invention in block diagram form;

FIG. 2 shows a signal portion characteristic of a heartbeat in an ECG signal;

FIG. 3 shows an example of ECG signal;

FIG. 4 shows an example of a PPG signal;

FIG. 5 shows an example of tachogram of an RR series;

FIG. 6 shows an example of a decision tree.

### DETAILED DESCRIPTION

With reference to the particular embodiment variant of FIG. 1, a drowsiness detection system of the invention comprises:
a module 1 for acquiring a cardiac signal 1a of an individual;
a module 2 for processing this cardiac signal 1a, which has the main function of detecting the time intervals between successive heartbeats in the cardiac signal 1a, and which delivers as output a chronological series RR consisting of a succession of samples RRᵢ, each RRᵢ sample quantifying a time interval between two successive heartbeats;
an extraction module 3, which is adapted to extract several HRV variables from the successive time intervals RR provided by the processing module 2;
a calculation module 4, which is adapted to calculate one or several direction aggregates and one or several shape aggregates from the HRV variables provided by the extraction module 3;
a drowsiness detection module 5, which is adapted to detect a drowsiness state from only said aggregates provided by the calculation module 4;
an alert module 6.

The detection system of the invention can advantageously be used to detect drowsiness, and preferably to detect the onset of drowsiness of a vehicle driver early on, and if drowsiness is detected, to issue an alarm, in order to warn the driver that their vigilance is reduced. However, the invention is not limited to this application alone, as the detection system can be used in all applications where it is useful to detect the drowsiness of an individual, and preferably of an individual in a static position.

In the context of the invention, the different modules 1 to 6 can be integrated into the same detection device, for example in the passenger compartment of a vehicle, for locally acquiring the cardiac signal and locally detecting the individual's drowsiness. In other variants of the invention, the module(s) 2, 3, 4 or 5 can be remote from the acquisition site of the cardiac signal. For example, the acquisition module 1 can be designed to remotely communicate the cardiac signal 1a, via any type of telecommunications network, to a remote processing assembly comprising the modules 2 to 5.

The technology used to produce modules 1 to 6 is not limiting to the invention. For example, and in a non-exhaustive manner, all of modules 1 to 6 can be implemented by means of one or several electronic processing units comprising one or several microprocessors or one or several microcontrollers or by means of one or several electronic processing units implemented in the form of a programmable circuit, for example of the FPGA type, or in the form of a specific electronic circuit of the ASIC type. Modules 3, 4 and 5 can also be implemented in the form of one or several software programs, which can be executed by a remote computer or server communicating remotely with the signal acquisition module 1.

### Acquisition Module 1—Cardiac Signal 1a

The acquisition module 1 comprises one or several sensors which are adapted to acquire a cardiac signal 1a of an individual.

In the context of the invention, the type of sensor(s) is of no importance.

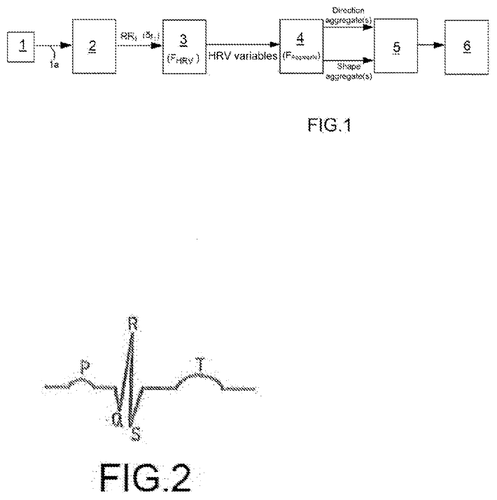

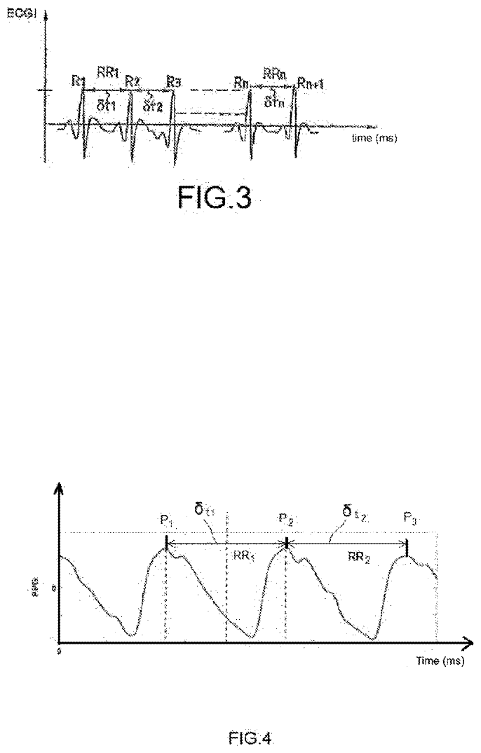

The sensors may, for example, and in a way that does not limit the invention, be a set of electrodes delivering a cardiac signal 1a of the ECG signal type (FIG. 3), which is representative of the individual's cardiac activity, and which comprises, in a known manner, for each heartbeat, five characteristic electrical waves P, Q, R, S, T, as illustrated in FIG. 2:
Wave P corresponds to depolarization of the earcups, and which exhibits a low amplitude and a dome shape;
the PQ space reflects the atrioventricular conduction time;
Wave R is considered in practice to be a marker of the ventricular systole, or heartbeat, the complex QRS reflecting ventricular contraction, and
Wave T reflects ventricular repolarization.

In a way that does not limit the invention, a sensor of the acquisition module may also be a cardiac pulse sensor, of the pulse oximeter or plethysmography sensor type, delivering a cardiac signal 1a, of the PPG (photoplethysmography) signal type having for example the profile of the signal of FIG. 4.

In the context of the invention, the sensor(s) of the acquisition module 1 can be integrated into a detection device carried by the individual, in the form, for example and non-exhaustively, of a bracelet, a ring or even a clamp.

The sensor(s) of the acquisition module 1 can also be integrated into an element manipulated by the individual, such as for example the steering wheel of a vehicle.

The sensor(s) of the acquisition module 1 can also be integrated into a garment worn by the individual.

The sensor(s) of the acquisition module 1 can also be integrated into the immediate environment of the individual and be for example integrated into the seat of a vehicle.

Regardless of the type of sensor, the cardiac signal 1a delivered by the acquisition module 1 can be an analog signal or a digital signal.

When the cardiac signal 1a is digital, the acquisition module 1 integrates an analog/digital converter making it possible to digitize the cardiac signal with a predetermined sampling frequency (fc), equal for example to 256 Hz.

When the cardiac signal 1a is analog, the processing module 2 generally comprises at the input said analogue/digital converter making it possible to digitize the cardiac signal 1a before detection by the processing module 2.

### Processing Module 2—RRᵢ Samples

The function of module 2 is, in a manner known per se, to detect the time intervals between successive heartbeats in the cardiac signal 1a.

This detection is preferably carried out in real time during the acquisition of the cardiac signal 1a.

When the cardiac signal is an ECG signal (FIG. 3), module 2 is preferably adapted to detect each time interval δtᵢ between two successive waves R (FIG. 3) and to construct, at the output of the module, a chronological series, called series “RR” and consisting of a succession of samples RRᵢ, the value of each RR sample being equal to the time interval δtᵢ between two successive waves Rᵢ, Rᵢ₊₁.

However, this does not limit the invention. In the case of a cardiac signal of the ECG type, module 2 can also be adapted to construct said RR series using the other depolarization waves (P, Q, S or T) of the ECG signal, the accuracy however being less good than using the waves R of the ECG signal.

When the cardiac signal is a cardiac pulse signal of the type of that of FIG. 4, the processing module 2 is preferably adapted to detect each time interval δtᵢ between two successive peaks Pᵢ, Pᵢ₊₁ (FIG. 4) characteristic of two successive cardiac pulses and for constructing, at the output of the module, a chronological series consisting of a succession of samples RRᵢ, the value of each sample RRᵢ being equal to the time interval δtᵢ between two successive peaks Pᵢ, Pᵢ₊₁.

In the present text, regardless of the type of cardiac signal 1a, the chronological series consisting of a succession of samples RRᵢ is referred to as “series RR”, whose value is equal to the time interval δtᵢ between two successive heartbeats of the cardiac signal 1a.

By way of illustration only, one example tachogram of a series RR as a function of time is shown in FIG. 5.

Optionally, module 2 can also comprise, in a manner known per se, one or several filters making it possible to filter the cardiac signal 1a delivered by the acquisition module 1 or to filter the series RR before supplying it to the extraction module 3, in order for example to eliminate and/or correct any artifacts present in the cardiac signal 1a.

### Extraction Module 3—HRV Variables

The extraction module 3 makes it possible to extract several HRV variables from a series RR provided by the processing module 2.

In the present text, the term “HRV variable” generally means any variable characteristic of the variation of the heart rate and calculated from a plurality of time intervals δtᵢ.

This extraction is preferably carried out in real time during the acquisition of the cardiac signal 1a.

Generally, each HRV variable is calculated from several samples RRᵢ of the series RR.

More particularly, each HRV variable is calculated from several successive samples RR taken over a predefined time interval T_{HRV}.

More particularly, from a practical point of view, each HRV variable is calculated from several successive samples RRᵢ in a first sliding time window F_{HRV} (FIG. 5) of width L_{HRV} predefined corresponding to this time interval T_{HRV}, or in other words, to a predefined number of samples RRᵢ taken into account in the sliding time window F_{HRV}.

To implement this sliding time window F_{HRV}, a FIFO-type shift register can for example be implemented.

The width L_{HRV} of the sliding time window F_{HRV} may be different from one HRV variable to the other, or may be the same for several HRV variables, or may be the same for all the HRV variables.

The wider the sliding time window F_{HRV} is, the larger the number of RRᵢ samples taken into account to calculate the corresponding HRV variable in this sliding time window F_{HRV} is.

The sliding interval of the sliding time window F_{HRV} may be an interval of a single sample RRᵢ or an interval of a plurality of samples RRᵢ.

Preferably, this sliding interval of the sliding time window F_{HRV} is less than the width L_{HRV} of the sliding time window F_{HRV}, that is less than the number of samples RRiᵢ in the sliding time window F_{HRV}.

The sliding interval of the sliding time window F_{HRV} may be different for each HRV variable or may be the same for several HRV variables or may be the same for all the HRV variables.

In the context of the invention, the HRV variables are preferably selected from those listed below, it being specified however that the invention is not limited to these particular examples of HRV variables.

### List of Preferential HRV Variables

In the time domain, the preferential HRV variables for performing the invention are: HRₘₑₐₙ, RMSSD, VCT, VLT, SDNN, CSI.

### HRₘₑₐₙ

This variable HRₘₑₐₙ is representative of the average of the heart rate in the sliding time window F_{HRV}.

This variable HRₘₑₐₙ expressed in beats/minute can in a known manner be calculated from a plurality of samples RR (δtᵢ) in the sliding time window F_{HRV} mentioned above by means of the following formula:

$$HRmean ( \frac{beats}{minute} ) = \frac{60 \cdot N}{{\sum }_{i = 1}^{N} δ {t}_{i}}$$

wherein:
N is the number of samples RR (δtᵢ) in the sliding window F_{HRV}
the time intervals δtᵢ are expressed in seconds.

### RMSSD

This HRV variable is the square root of the average of the squared differences between the successive samples RRᵢ(δtᵢ) in the sliding time window F_{HRV}.

### VCT (Short-Term Variability)

This HRV variable was to date developed specifically for the analysis of the heart rate of fetuses and makes it possible to analyze the short-term variability of the heart rate.

VCT is calculated on a series of RRᵢ(δtᵢ) resampled at 4 hz. It analyses the average heart rate differences between two successive short epochs (for example of 3.75 s) successive in a sliding time window F_{HRV} of one minute (that is 16 epochs). VCT then represents the average of the absolute value of these differences divided by 2.

It can be calculated by means of the following formula:

$$VCT = \frac{1}{2 \times 16} \times {\sum }_{i = 2}^{16} abs ( {X}_{i} - {X}_{i - 1} )$$

wherein Xᵢ₋₁ and Xᵢ represent the average heart rate respectively on two successive short epochs (for example of 3.75 s).

### VLT (Long-Term Variability)

This HRV variable was to date developed specifically for the analysis of the heart rate of fetuses and makes it possible to analyze the long-term variability of the heart rate.

As for variable VCT, variable VLT is calculated on a series of RRᵢ(δtᵢ) resampled at 4 hz.

VLT represents the difference between the minimum value and the maximum value of the average heart rates Xᵢ calculated from successive epochs (for example of 3.75 s) in a sliding time window F_{HRV} of a minute.

### SDNN

This HRV variable is the standard deviation of the samples RRᵢ(δtᵢ) in the sliding time window F_{HRV}.

It can be calculated by means of the following formula:

$$SSNN = \sqrt{\frac{1}{N} \times {\sum }_{i = 1}^{N} {( δ {t}_{i} - M )}^{2}}$$

wherein:
N is the number of RRᵢ (δtᵢ) samples in the sliding window F_{HRV}
M is the average of the time intervals δtᵢ in the sliding window F_{HRV}.

### CSI

This HRV variable is calculated by means of the formula:

CSI=SD2²/SD1

wherein:

$$S D 1 = \frac{\sqrt{2}}{2} S D [ δ {t}_{i} - δ {t}_{i + 1} ]$$

$$SD 2 = \sqrt{2 S {D [ δ {t}_{i} ]}^{2} - \frac{1}{2} S {D [ δ {t}_{i} - δ {t}_{i + 1} ]}^{2}}$$

and

SD [δtᵢ−δtᵢ₊₁] is the standard deviation of the differences between successive time intervals δtᵢet δtᵢ₊₁ in the sliding time window F_{HRV}.

In the frequency domain, the preferred HRV variables for the invention are the variables commonly designated HF, LF, HF/LF, LF/HF.

In general, to obtain an HRV variable in the frequency domain, in particular HF, LF, HF/LF, LF/HF, an additional intermediate step of transformation into the frequency domain of the time signal RR is carried out, for example, and in a non-exhaustive manner, by means of a fast Fourier transform (FFT), a wavelet transform or an autoregressive model (ARMA) and a signal in the frequency domain is obtained, on which the calculation of the HRV variables, in particular HF, LF, HF/LF, LF/HF is carried out.

In a known manner, the HF and LF variables characterize the spectral power density or the spectral power of the series of RRᵢ samples in the sliding time window F_{HRV} aforementioned, in a high frequency band, preferably between 0.15 Hz and 0.4 Hz for the HF variable and in a low frequency band, preferably between 0.04 Hz and 0.15 Hz for the LF variable.

These HF and LF variables are, in a known manner, obtained from an integration of the signal (that is from the calculation of the “area under the curve” of the signal) from the transformation of the time signal RR into the frequency domain, this integration being carried out in a frequency band which is specific and different for each variable, and preferably:
for the variable HF: frequency band between 0.15 Hz and 0.4 Hz;
for the variable LF: frequency band between 0.004 Hz and 0.15 Hz.

### Other Variable: HR (Instantaneous Heart Rate)

Optionally, in addition to the HRV variables characteristic of the variability of the heart rate and each calculated from several samples RRᵢ, the extraction module 3 can also, in certain variant embodiments, calculate and provide to the calculation module 4 an additional variable called HR, which is not a HRV variable within the meaning of the present text and of the invention, which is calculated from a single sample RRᵢ, and which is characteristic of the instantaneous heart rate.

This HR variable expressed for example in number of heartbeats per minute can in a known manner be calculated from a single sample RRᵢ expressed in seconds by means of the following formula:

HR(beats/minute)=60/δtᵢ

### Calculation Module 4—Aggregates

Module 4 for calculating the aggregates is adapted to calculate at least one direction aggregate for one or several HRV variables or at least one shape aggregate for one or several HRV variables.

Preferably, module 4 for calculating the aggregates is adapted to calculate at least one direction aggregate for one or several HRV variables and at least one shape aggregate for one or several HRV variables.

In certain alternative embodiments, the shape aggregate(s) are calculated for HRV variables different from those used for the calculation of the direction aggregate(s).

In certain alternative embodiments, a direction aggregate and a shape aggregate can be calculated for the same HRV variable.

This calculation of aggregates is preferably carried out in real time during the acquisition of the cardiac signal 1a.

Generally, each direction aggregate and/or each shape aggregate is calculated for a HRV variable from several successive discrete values HRVᵢ of the HRV variable in a second sliding time window F_{Aggregate} of predefined width L_{Aggregate}.

This width L_{Aggregate} of the sliding time window F_{Aggregate} corresponds to a time interval T_{Aggregate} or in other words to a predefined number of discrete HRVᵢ values taken into account in the sliding time window F_{Aggregate}.

This width L_{Aggregate} of the sliding time window F_{Aggregate} may be different from one variable to another, and if applicable for a same variable may be different for a direction aggregate and for a shape aggregate.

Nevertheless, in a preferred variant embodiment, all the aggregates (shape and direction aggregate(s)) will be calculated for all the HRV variables with the same sliding time window F_{Aggregate}.

The wider the sliding time window F_{Aggregate} is, the larger the number of samples of the HRV variable taken into account for the calculation of the shape or direction aggregate in this sliding time window F_{Aggregate} is.

More particularly, but in a way that does not limit the invention, the time interval T_{Aggregate} corresponding to the width of the sliding time window F_{Aggregate} will preferably be at least 30 seconds and preferably less than or equal to 10 minutes.

The sliding interval of the sliding time window F_{Aggregate} may in the case be an interval of a single sample of the HRV variable or an interval of a plurality of samples of the HRV variable.

Preferably, this sliding interval of the sliding time window F_{Aggregate} is less than the width L_{Aggregate} of the sliding time window F_{Aggregate}, that is less than the number of samples of the HRV variable in the sliding time window F_{Aggregate}.

The sliding interval of the sliding time window F_{Aggregate} may be the same for all aggregates or may be specific to an aggregate.

Preferably, but optionally, in order to facilitate the adaptation of the drowsiness detection method of the invention to different types of applications, the width of the sliding time window F_{Aggregate} and/or the sliding interval of the sliding time window F_{Aggregate} are adjustable.

To implement this sliding time window F_{Aggregate}, a FIFO-type shift register can for example be implemented.

Optionally, when the extraction module 3 is adapted to also calculate the aforementioned HR variable, in this case the calculation module 4 can also calculate a direction aggregate for the HR variable and/or an shape aggregate for the HR variable from the discrete values (samples) HRᵢ taken by this HR variable in a sliding time window F_{Aggregate}, and in particular from several successive discrete values HRᵢ of the HR variable in a sliding time window F_{Aggregate} of predefined width L_{Aggregate}.

### Direction Aggregates

In general, a direction aggregate is a variable, which is calculated for a HRV variable, and optionally for the HR variable, from discrete values (samples) of the HRV (or HR) variable, in the sliding time window F_{Aggregate}, and which defines the trend (upward, downward, constant) of the HRV (or HR) variable in the sliding time window F_{Aggregate}.

Preferably, and more particularly, a direction aggregate is a variable whose sign defines whether the trend of the HRV (or HR) variable in the sliding time window F_{Aggregate} is downward or upward, and preferably which is zero when the trend of the HRV (or HR) variable in the sliding time window F_{Aggregate} is constant.

Preferably, but not necessarily, the absolute value of a direction aggregate quantifies said trend of the HRV (or HR) variable in the sliding time window F_{Aggregate}.

Preferably, the direction aggregates can be selected from the three particular types of direction aggregates (“DIRECTION”, “DELTA”, “LINEAR REGRESSION”) detailed below, however, it is specified that the invention is not limited to those particular examples of direction aggregates.

### “Direction”

This aggregate is obtained by calculating the difference between the last and the first value of the HRV (or HR) variable in the sliding time window F_{Aggregate}.

When this difference is positive, the trend of the HRV (or HR) variable is upward. When this difference is negative, the trend of the HRV (or HR) variable is downward.

### “Delta”.

The absolute value of this aggregate is obtained by calculating the difference between the maximum value of the HRV (or HR) variable and the minimum value of the HRV (or HR) variable in the sliding time window F_{Aggregate}. The sign of this aggregate is for example obtained from the chronological position of said maximum value of the variable relative to the chronological position of said minimum value of the variable in the sliding time window F_{Aggregate}. When the chronological position in the sliding time window F_{Aggregate} of the maximum value of the HRV (or HR) variable is subsequent to the minimum value of the HRV (or HR) variable, the sign of the direction aggregate DELTA is positive, and the trend of the HRV (or HR) variable is upward. When the chronological position in the sliding time window F_{Aggregate} the maximum value of the HRV (or HR) variable is prior to the minimum value of the HRV (or HR) variable, the sign of the direction aggregate DELTA is negative, and the trend of the HRV (or HR) variable is downward.

### “Linear Regression”

This aggregate is obtained by calculating, by linear regression, for example by using the least squares method, the straight line approximating all the values of the HRV (or HR) variable in the sliding time window F_{Aggregate}, the aggregate “LINEAR REGRESSION” being the slope (leading coefficient) of this straight line. When this slope is positive, the trend of the HRV (or HR) variable is upward. When this slope is negative, the trend of the HRV (or HR) variable is downward.

### Shape Aggregates

In general, a shape aggregate is a variable which is calculated from discrete values (samples) of the HRV (or HR) variable in the sliding time window F_{Aggregate} and that quantifies the shape of a distribution of the samples (values) of the HRV (or HR) variable in the sliding time window F_{Aggregate}.

Preferably, the shape aggregates can be selected from the different particular types of shape aggregates detailed below, it being specified, however, that the invention is not limited to those particular examples of shape aggregates.

### Standard Deviation

The standard deviation measures in a manner known per se the dispersion of the distribution of a variable.

In the context of the invention, the standard deviation (std) of the HRV (or optionally HR) variable in the sliding time window F_{Aggregate} can be calculated by means of the following formula:

$$std = \sqrt{\frac{{Σ}_{i = 1}^{N} {( {x}_{i} - {x}_{m e a n} )}^{2}}{N - 1}}$$

wherein:
N is the number of values (samples) of the HRV (or HR) variable in the sliding time window F_{Aggregate},
xₘₑₐₙ is the mean of the values of the HRV (or HR) variable in the sliding time window F_{Aggregate};
xᵢ is the value of the HRV (or HR) variable at the i-th position in the sliding time window F_{Aggregate};

### Kurtosis

Kurtosis, also commonly called an acuity coefficient or flattening coefficient, measures in a manner known per se the acuity of the distribution of a variable.

There are several known methods for calculating the Kurtosis of a variable.

In the context of the invention, the Kurtosis of the HRV variable in the sliding time window F_{Aggregate} can for example be calculated by means of the following formula:

$$Kurtosis = \frac{( N + 1 ) N}{( N - 1 ) ( N - 2 ) ( N - 3 )} \times \frac{{\sum }_{i = 1}^{N} {( {x}_{i} - {x}_{m e a n} )}^{4}}{{k}_{2}^{2}} - 3 \frac{{( N - 1 )}^{2}}{( N - 2 ) ( N - 3 )}$$

wherein:
N is the number of values (samples) of the HRV (or HR) variable in the sliding time window F_{Aggregate};
xₘₑₐₙ is the mean of the values of the HRV (or HR) variable in the sliding time window F_{Aggregate};
xᵢ is the value of the HRV (or HR) variable at the i-th position in the sliding time window F_{Aggregate};
k₂ is the variance of the samples of the HRV (or HR) variable in the sliding time window F_{Aggregate}.

### Skewness

Skewness, also commonly called an asymmetry coefficient, measures in a manner known per se the asymmetry of the distribution of a variable.

There are several known methods for calculating the Skewness of a variable.

In the context of the invention, the Skewness of the HRV variable in the sliding time window F_{Aggregate} can for example be calculated by means of the following formula:

$$Skewness = \frac{\sqrt{N ( N - 1 )}}{N - 2} \times \frac{{\sum }_{i = 1}^{N} {( {x}_{i} - {x}_{m e a n} )}^{3}}{N \cdot {s}^{3}}$$

wherein:
N is the number of values (samples) of the HRV (or HR) variable in the sliding time window F_{Aggregate};
xₘₑₐₙ is the mean of the values ᵢ of the HRV (or HR) variable in the sliding time window F_{Aggregate};
xᵢ is the value of the HRV (or HR) variable at the i-th position in the sliding time window F_{Aggregate};
S is the standard deviation of the samples of the HRV (or HR) variable in the sliding time window F_{Aggregate}.

Other shape aggregates can also be derived from the shape aggregate(s) mentioned above.

For example and in a non-exhaustive manner, aggregates having a shape derived from the standard deviation (std) can be calculated, such as for example M/std or std/M, M being the average of HRVᵢ values (or HRᵢ) of the HRV (or HR) variable in the sliding time window F_{Aggregate}.

Preferably, the invention can be carried out on the one hand by using at least two different HRV variables, preferably at least three different HRV variables, and more preferably still at least four selected.

It is preferable to use several HRV variables to make the detection of drowsiness even more reliable and more universal.

Preferably, for a better detection of the drowsiness, the extraction module 3 is adapted to extract at least one HRV variable in the time domain and at least one HRV variable in the frequency domain.

Preferably, the HRV variable(s) are chosen from the list of preferential HRV variables mentioned above (HRₘₑₐₙ, RMSSD, VCT, VLT, SDNN, CSI, HF, LF, HF/LF), and module 4 is adapted to calculate, for one or several of these HRV variables, one or several direction aggregates preferably chosen from the list of the aforementioned direction aggregates (“DIRECTION”, “DELTA”, “LINEAR REGRESSION”) and to calculate, for one or several of these HRV variables, one or several shape aggregates preferably chosen from the list of aggregates of the aforementioned form (std, Kurtosis, Skewness, M/std or std/M).

As preferential but not limiting and non-exhaustive examples of the invention, the trend of certain preferential aggregates for certain HRV variables, before the appearance of drowsiness, has been summarized in both Tables A and B, this trend being able to be analyzed by the drowsiness detection algorithm in order to detect drowsiness of an individual as early as possible.

TABLE A
Aggregates - HRV Variables in the time domain

| | Trend in |
|---|---|
| | aggregates before |
| HRV Variables - Aggregates | drowsiness |
| HRmean | |
| DIRECTION, LINEAR | Downward or |
| REGRESSION, DELTA | Constant Trend |
| Absolute Value of DELTA | Decreases |
| Kurtosis | Decreases |
| Skewness | Decreases |
| Std | Decreases |
| Std/M | Decreases |
| RMSSD | |
| DIRECTION, LINEAR | Upward or Constant |
| REGRESSION, DELTA | Trend |
| Absolute Value of DELTA | Decreases |
| Skewness | Increases |
| Std | Decreases |
| Std/M | Decreases |
| SDNN | |
| DIRECTION, LINEAR | Upward or Constant |
| REGRESSION, DELTA | Trend |
| Absolute Value of DELTA | Decreases |
| Std | Decreases |
| VCT | |
| DIRECTION, LINEAR | Constant Trend |
| REGRESSION, DELTA | |
| Absolute Value of DELTA | Decreases |
| Std | Decreases |
| Std/M | Decreases |
| VLT | |
| DIRECTION, LINEAR | Upward trend |
| REGRESSION, DELTA | |
| Absolute Value of DELTA | Decreases |
| Skewness | Decreases |
| Std | Decreases |
| Std/M | Decreases |
| CSI | |
| Std/M | Increases |

TABLE B
Aggregates - HRV Variables in the frequency domain

| | Trend in |
|---|---|
| | aggregates before |
| HRV Variables - Aggregates | drowsiness |
| HF | |
| DIRECTION, LINEAR | Constant Trend |
| REGRESSION, DELTA | |
| Absolute Value of DELTA | Decreases |
| Kurtosis | Decreases |
| Skewness | Decreases |
| Std | Decreases |
| Std/M | Decreases |
| LF | |
| DIRECTION, LINEAR | Constant Trend |
| REGRESSION, DELTA | |
| Absolute Value of DELTA | Decreases |
| Kurtosis | Decreases |
| Skewness | Decreases |
| Std | Decreases |
| Std/M | Decreases |
| LF/HF | |
| DIRECTION, LINEAR | Constant Trend |
| REGRESSION, DELTA | |
| Absolute Value of DELTA | Decreases |
| Skewness | Increases |
| Std | Decreases |
| Std/M | Decreases |

The use of HRV variables and direction aggregate(s) calculated for one or several of these HRV variables and/or of shape aggregate(s) calculated for one or several of these HRV variables advantageously makes it possible to perform reliable drowsiness detection, without it being essential to use other drowsiness detection devices or to use in addition physiological signals other than the cardiac signal 1a.

The drowsiness detection system of the invention may therefore advantageously be used for the detection of drowsiness, based solely on the direction aggregate(s) and/or the shape aggregate(s), without requiring other detection devices or without requiring the acquisition of physiological signals other than the cardiac signal 1a.

Nevertheless, in the context of the invention, the drowsiness detection system of the invention may also be used in addition to other known detection devices, such as for example detection devices based on the analysis of eye blinking, detection devices based on individual behavioral analysis, detection devices based on the analysis of vehicle movements, or drowsiness detection devices using physiological signals other than a cardiac signal.

Furthermore, compared to the drowsiness detection solutions that directly use the HRV variables to detect drowsiness, the use according to the invention of HRV variables and direction aggregate(s) calculated for one or several of these HRV variables and/or of shape aggregate(s) calculated for one or several of these HRV variables can advantageously make it possible to carry out drowsiness detection which is more universal, that is to say which is not dependent on or specific to an individual.

Moreover, the use according to the invention of HRV variables and direction aggregate(s) calculated for one or several of these HRV variables and/or of shape aggregate(s) calculated for one or several of these HRV variables can advantageously make it possible, in many cases, to carry out a detection of early drowsiness, that is to say to detect an onset of drowsiness, well before the phase in which the individual is asleep.

Preferably, but not necessarily, the detection of drowsiness is carried out by preferentially using as HRV variables, at least the variables LF, HF, HF/LF, LF/HF, and more preferentially by calculating at least one direction aggregate for each of these HRV variables.

Preferably, but not necessarily, the detection of drowsiness is carried out by preferentially using as HRV variables, at least the variables HF and HRₘₑₐₙ, and by calculating at least one shape aggregate for each of these HRV variables or by preferentially using the HR variable and several HRV variables including at least the variable HF and by calculating at least one shape aggregate for each of these variables HR and HF.

Preferably, but not necessarily, the detection of drowsiness is carried out using a plurality of HRV variables, including at least preferentially the HF variable, and by calculating at least one direction aggregate and at least one shape aggregate for this HF variable.

### Drowsiness Detection Module 5

The different shape aggregate(s) and/or direction aggregate(s) are provided as input variables to the drowsiness detection module 5.

Generally, the drowsiness detection module 5 executes a detection algorithm that allows the individual's drowsiness to be detected from these aggregates.

This drowsiness detection is carried out in real time during the acquisition of the cardiac signal 1a.

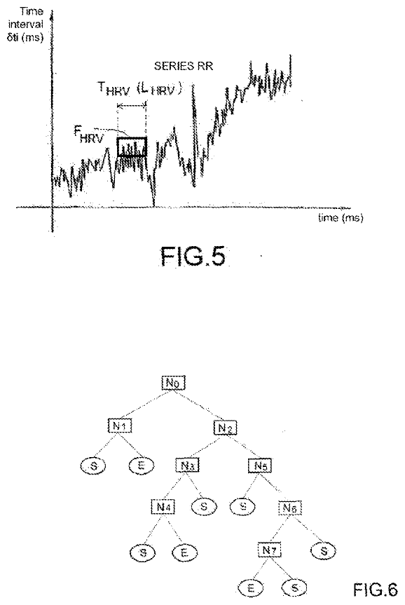

More particularly, but not necessarily, the detection by module 5 is carried out by using the direction aggregate and/or the shape aggregate as test variables in a decision tree, such as for example that of FIG. 6, with an automatic classification, at the output of the decision tree, of the cardiac signal as being characteristic (S) of a drowsiness of the individual or not characteristic (E) of a drowsiness of the individual.

At the root (N0) of the detection tree and at each node (N1, N2, N3, . . . ) of the decision tree at least one of the aggregates coming from the aggregate calculation module 4 (shape aggregate or direction aggregate) is compared to a predefined threshold (S0, S1, S2, . . . ).

The particular structure of the decision tree of FIG. 6 is provided by way of example only and is not limiting on the structures of decision trees that can be implemented.

The predictive algorithm for the detection of drowsiness can also implement the known automatic learning technique called “random forest”, which performs training on multiple decision trees trained on different data subsets (HRV variables and direction aggregate(s) and shape aggregate(s)).

Although the implementation of an algorithm based on one or several decision trees is preferential, the invention can nevertheless also be implemented by using other types of predictive algorithms, such as for example and non-exhaustively, an algorithm based on a neural network, a convolutional neural network, a logistic regression, or any other artificial intelligence model.

### Alert Module 6

The drowsiness detection module 5 communicates with the alert module 6 in order to keep it informed, in real time, and during the acquisition of the cardiac signal 1a, of whether the individual is drowsy or not.

The alert module 6 is adapted to automatically trigger an action, as soon as it is informed of a state of drowsiness of the individual by the drowsiness detection module 5.

This action is for example the triggering of a visual and/or audible and/or mechanical alarm signal (for example vibrations) in the individual's environment, so as to warn at least the individual of their state of drowsiness, in order for the individual to take measures (for example, pausing driving and taking a rest) necessary to restore their vigilance.

1. A method for detecting an individual's drowsiness including acts comprising:
(a) acquiring a cardiac signal of the individual by using at least one sensor,
(b) processing the cardiac signal allowing the detection of the time intervals between successive heartbeats,
(c) extracting, from the time intervals between successive heartbeats, one or several different HRV variables that are characteristics of the heart rate variability, each HRV variable being obtained from a plurality of time intervals between successive heartbeats,
(d) calculating at least one direction aggregate for one or several of said HRV variables, each direction aggregate being a variable, which is calculated from values of the HRV variable in a sliding time window, and which characterizes a trend of the HRV variable in this sliding time window, and/or calculating at least one shape aggregate for one or several of said HRV variables, each shape aggregate being a variable, which is calculated from the values of the HRV variable in a sliding time window and quantifying the shape of a distribution of the values of the HRV variable in this sliding time window, and
(e) processing the direction aggregate(s) and/or the shape aggregate(s) by a detection algorithm for detecting the individual's drowsiness.

2. The method according to claim 1, wherein acts (b) to (e) are carried out during act (a) of acquiring the cardiac signal.

3. The method according to claim 1, wherein at least two different HRV variables are extracted in act (c).

4. The method according to claim 1, wherein in act (c) at least one HRV variable in the time domain and at least one HRV variable in the frequency domain are extracted.

5. The method according to claim 1, wherein the HRV variable(s) are selected from the HRV variables of the following list: Heart Rate Mean (HRₘₑₐₙ), Root Mean Square of Successive Differences (RMSSD), Short Term Variability (VCT), Long Term Variability (VLT), Standard Deviation Normal to Normal (SDNN), Cardio Stress Index (CSI), High Frequency power or power density (HF), Low Frequency power or power density (LF), HF/LF, LF/HF.

6. The method according to claim 1, wherein in act (c) at least variables Low Frequency power or power density (LF) and High Frequency power or power density (HF) are extracted, and in act (d) at least one direction aggregate for each of these variables is calculated.

7. The method according to claim 1, wherein in act (c) at least variables High Frequency power or power density (HF) and Heart Rate Mean (HRₘₑₐₙ) are extracted, and in act (d) at least one shape aggregate for each of these variables is calculated.

8. The method according to claim 1, wherein in act (c) several HRV variables are extracted, including at least the variable High Frequency power or power density (HF), and in act (d) at least one direction aggregate and at least one shape aggregate are calculated for the variable HF.

9. The method according to claim 1, wherein in act (c) an HR variable characteristic of the instantaneous heart rate calculated from a single time interval between two successive heartbeats is also extracted, and wherein in act (d) at least one direction aggregate is calculated from the values of the HR variable in a sliding time window, said direction aggregate characterizing the trend of the HR variable in this sliding time window and/or at least one shape aggregate, from the values of this HR variable in a sliding time window, said shape aggregate quantifying the shape of a distribution of the values of the HR variable in this sliding time window.

10. The method according to claim 9, wherein in act (c), the HR variable and several HRV variables including at least the HF variable are extracted, and in act (d) at least one shape aggregate for each of these HR and HF variables is calculated.

11. The method according to claim 1, wherein a direction aggregate is a variable whose sign defines whether the trend of the variable in said sliding time window is downward or upward.

12. The method according to claim 1, wherein at least one of the direction aggregates is calculated from the difference between the last and the first value of the variable in the sliding time window.

13. The method according to claim 1, wherein a sign of at least one direction aggregate is calculated from a chronological position of a maximum value of the variable in the sliding time window relative to a chronological position of a minimum value of the variable in the sliding time window.

14. The method according to claim 1, wherein at least one of the direction aggregates is calculated from a slope of a straight line obtained by linear regression on values of the variable in the sliding time window.

15. The method according to claim 1, wherein at least one of the shape aggregates is calculated from at least one of the following coefficients: acuity coefficient (Kurtosis) of a distribution of the variable, asymmetry coefficient (Skewness) of a distribution of the variable, standard deviation (std) of the variable.

16. The method according to claim 1, wherein a width of the sliding time window corresponds to a time interval of at least 30 seconds.

17. The method according to claim 1, wherein a width of the sliding time window corresponds to a time interval less than or equal to 10 minutes.

18. The method according to claim 1, wherein a width of the sliding time window is adjustable.

19. The method according to claim 1, wherein the calculation of each direction aggregate is carried out with the same sliding time window.

20. The method according to claim 1, wherein the calculation of each shape aggregate is carried out with the same sliding time window.

21. The method according to claim 1, wherein the calculation of all direction and/or shape aggregates is carried out with the same sliding time window.

22. The method according to claim 1, wherein a sliding interval of the sliding time window is adjustable.

23. The method according to claim 1, wherein act (e) comprises using the direction aggregate(s) and the shape aggregate(s) as test variables in at least one decision tree, with an automatic classification, at an output of the decision tree, of the cardiac signal as being characteristic of drowsiness of the individual or not characteristic of drowsiness of the individual.

24. The method according to claim 1, wherein in act (c), each HRV variable is obtained in the time or frequency domain from several time intervals between successive heartbeats in a sliding time window.

25. A system for detecting drowsiness including:
at least one electronic processing unit; and
at least one non-transitory computer readable medium comprising instructions stored thereon which when executed by the at least one processing unit configures the at least one processing unit to implement a method of detecting an individual's drowsiness, the method comprising:
(a) acquiring a cardiac signal of the individual by using at least one sensor,
(b) processing the cardiac signal allowing the detection of the time intervals between successive heartbeats,
(c) extracting, from the time intervals between successive heartbeats, one or several different HRV variables that are characteristics of the heart rate variability, each HRV variable being obtained from a plurality of time intervals between successive heartbeats,
(d) calculating at least one direction aggregate for one or several of said HRV variables, each direction aggregate being a variable, which is calculated from values of the HRV variable in a sliding time window, and which characterizes a trend of the HRV variable in the sliding time window, and/or calculating at least one shape aggregate for one or several of said HRV variables, each shape aggregate being a variable, which is calculated from the values of the HRV variable in a sliding time window and quantifying the shape of a distribution of the values of the HRV variable in the sliding time window, and
(e) processing the direction aggregate(s) and/or the shape aggregate(s) by a detection algorithm for detecting the individual's drowsiness.

26. (canceled)

27. A non-transitory computer readable medium comprising program code instructions stored thereon which when executed by one or several electronic processing units, cause the one or several electronic processing units to implement a method of detecting an individual's drowsiness, comprising:
(a) acquiring a cardiac signal of the individual by using at least one sensor,
(b) processing the cardiac signal allowing the detection of the time intervals between successive heartbeats,
(c) extracting, from the time intervals between successive heartbeats, one or several different HRV variables that are characteristics of the heart rate variability, each HRV variable being obtained from a plurality of time intervals between successive heartbeats,
(d) calculating at least one direction aggregate for one or several of said HRV variables, each direction aggregate being a variable, which is calculated from values of the HRV variable in a sliding time window, and which characterizes a trend of the HRV variable in the sliding time window, and/or calculating at least one shape aggregate for one or several of said HRV variables, each shape aggregate being a variable, which is calculated from the values of the HRV variable in a sliding time window and quantifying the shape of a distribution of the values of the HRV variable in the sliding time window, and
(e) processing the direction aggregate(s) and/or the shape aggregate(s) by a detection algorithm for detecting the individual's drowsiness.

28. (canceled)

29. The non-transitory computer readable medium according to claim 27 wherein the method further comprises calculating, in act (c), each HRV variable in the time or frequency domain from several time intervals between successive heartbeats in a sliding time window.
